(19) United States
(12) Patent Application Publication
(10) Pub. No.: US 2025/0099140 A1
(43) Pub. Date: Mar. 27, 2025

(54) MEDICAL IMPLANTS FOR GENERATING FUSION BETWEEN TWO BONES

(71) Applicant: ORTHOFUMDAMENTALS, LLC, Waltham, MA (US)

(72) Inventors: Matthew Palmer, Weston, MA (US); Jarred Sakakeeny, Hopkinton, MA (US)

(21) Appl. No.: 18/728,645

(22) PCT Filed: Jan. 12, 2023
(86) PCT No.: PCT/US2023/010699

§ 371 Date: Jul. 12, 2024

Related U.S. Application Data
(60) Provisional application No. 63/357,642, filed on Jul. 1, 2022.
(60) Provisional application No. 63/298,954, filed on Jan. 12, 2022.

(51) Int. Cl. A61B 17/70; A61B 17/86

(52) U.S. Cl. CPC A61B 17/7055; A61B 17/864

(57) ABSTRACT

Orthopedic implants for generating fusion between two bones are disclosed. Notably, the implants do not require supplemental fixation. Disclosed wedge implants may include an interlocking, e.g. dovetail feature. Screw-type implants are disclosed as well. Various surface modifications and structural features are disclosed to promote bone growth and fusion. Related kits and methods are also disclosed.

### CROSS-REFERENCE TO RELATED APPLICATIONS

This application claims the benefit of priority to U.S. Provisional Patent Application Ser. No. 63,298,954, filed on Jan. 12, 2022 and titled MEDICAL IMPLANTS FOR GENERATING FUSION BETWEEN TWO BONES, as well as to U.S. Provisional Patent Application Ser. No. 63/357,642, filed on Jul. 1, 2022 and titled MEDICAL SCREW IMPLANTS FOR GENERATING FUSION BETWEEN TWO BONES, the entire disclosure of each of which is hereby incorporated herein by reference in its entirety for all purposes.

### FIELD OF THE INVENTION

The present invention relates to devices for generating fusion between two or more bones to promote healing. The invention finds particular utility in the ability to transfix two or more bones with screws, spacers, or wedges designed with specific geometry to not require the use of supplemental fixation. While the invention has application throughout the body, its utility will be illustrated in the context of repair between two bony elements such as the pelvis and sacrum.

### BACKGROUND

Orthopedic implants are used for a variety of disorders including age related degeneration, trauma, congenital and idiopathic deformities as well as pathologic fractures.

Non-surgical treatments, such as medication, rehabilitation and exercise can be effective, however, may fail to relieve the symptoms associated with these disorders. Surgical treatment of these disorders includes correction, fusion, and fixation using implantable devices. As part of these surgical treatments, orthopedic constructs often use structural spacers, screws, cages or wedges to restore alignment and stabilize the surgical site in anticipation of healing or fusion.

In the field of orthopedics, it is common to join together two or more bones and hold them in place so as to minimize motion and promote healing or fusion across the bony elements. This is commonly accomplished using structural spacers, cages, wedges, or screws. Structural spacers, cages, or wedges (together referred to as “wedges”) are placed between two bony elements and act as a bridging element to provide structural support to the gap between the two bony elements while the two bones fuse. The adjacent bone may fuse onto, thru, or around the spacer. While wedges do promote fusion, they do not provide a method for maintaining the position of the bones or limiting their motion relative to each other while fusion occurs. Often times, a plate or screws are used as an additional means of supplemental fixation to stabilize the two bones until fusion occurs. Placement of additional fixation elements can be risky as they can be inadvertently placed into delicate anatomical structures such as neurologic or vascular structures. Additionally, supplemental fixation can become another source of infection or irritation to the patient. Thus, there exists a clinical need for wedge devices that can act as a spacer to promote fusion while also providing a means of stabilizing the two bones and limiting their motion, thus allowing for fusion to occur.

Alternatively, screws can be used to join together two or more bones and hold them in place so as to minimize motion and promote healing or fusion across the bony elements. Screws may have heads or may be headless. They may utilize a differential thread pitch to generate compression between bones and hold them in place during fusion. However, depending on the anatomy of the bones being fused, it may not be possible to transfix two bones with a screw without creating a large surgical opening or disturbing other tissue. Additionally, screws have a minimal surface area and are not optimized for bone to grow onto to aid with fusion. Thus, there exists a clinical need for screw devices that can transfix two bones while limiting anatomical disturbances and still promote fusion between two bones.

### SUMMARY

The present invention provides a novel orthopedic implant which can be placed between two adjacent bony elements to transfix the bones in place to enable healing.

The implant can be metallic (such as, but not limited to Titanium Alloy), polymeric (such as, but not limited to PEEK or PEKK) or comprised of allograft bone. The geometry of the disclosed implants enables the transfixing of two or more bones without the need for additional anchors (i.e. screws, nails, or plates). The implant is constrained by an upper and lower surface which contacts the adjoining bones. The center of the implant can be hollow or consist of a channel of less dense or porous material to encourage bony bridging between the two native bone elements. The implant can contain a central channel generally progressing longitudinally along the length of the implant. A plurality of openings or fenestrations along the surfaces create a pathway from inside of the implant to the surrounding anatomy and can be created with varying hole sizes, shapes or porosity as to influence the directionality of flow of material from inside the implant to the surrounding anatomy or as to influence preferentially healing and bony fusion into one particular surface of the implant. The central channel and accompanying fenestrations can accommodate bone graft material or therapeutic agents for delivery into the surrounding host tissue environment.

The top and bottom surface may be tapered as to promote distraction across the longitudinal axis as well as to provide ease of insertion. Additionally, the top and bottom surface may be more parallel in nature.

In one embodiment, a wedge-shaped implant is provided. The implant can be a multitude of shapes including, but not limited to a cylinder, a cube, or a rectangular cube, or other three-dimensional shapes. In a preferential embodiment, the wedge is characterized by at least one interlocking feature, e.g. dovetail feature as described herein, to transfix the two bones. The wedge includes a wall that defines an inner and outer surface. The inner surface defines a cavity with a plurality of openings or fenestrations. The openings are disposed along the length of the cage.

In one embodiment, the implant is a cylindrically shaped screw with a thread geometry which is designed to transfix two adjacent bone segments. The transfixing is accomplished using a novel thread geometry. The thread geometry is helically wound around the cylindrical implant and has a first surface which terminates at a first fixation control ramp having another surface sloping upwardly at a predetermined angle. This thread geometry is optimized to capture bone from the adjacent bone segments and resists both axial pull out and bony translation/rotation.

In one embodiment, the implant includes an implant receiver and a shaft. The shaft includes an indicator to identify the orientation of the openings of the implant for controlling the deployment of a flowable biologically active material.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C present schematic views showing a novel transfixing wedge formed in accordance with the present invention.

FIG. 2 is a schematic view showing the top of the novel transfixing wedge formed in accordance with the present invention.

FIG. 3 is a schematic view showing a cross-section of the novel transfixing wedge formed in accordance with the present invention.

FIG. 4 is a schematic view showing the sides of the novel transfixing wedge formed in accordance with the present invention.

FIG. 5 is a schematic view showing the novel transfixing wedge formed in accordance with the present invention implanted into bone.

FIG. 6 is a schematic view showing the placement of the novel transfixing wedge formed in accordance with the present invention within the sacroiliac joint.

FIG. 7 is a schematic view showing the placement of the novel transfixing wedge formed in accordance with the present invention between two adjacent vertebrae.

FIG. 8 is a schematic view showing a novel transfixing screw formed in accordance with the present invention.

FIG. 9 is a schematic view showing the cross-section of a novel transfixing screw formed in accordance with the present invention.

FIG. 10 is a schematic view showing the novel transfixing screw formed in accordance with the present invention engaging two bone fragments.

FIG. 11 is a schematic view showing the novel transfixing screw in accordance with the present invention transfixing the sacrum and ilium.

FIG. 12 is a schematic view of a kit in accordance with one or more embodiments.

FIG. 13 is a schematic view of an implant device in accordance with one or more embodiments.

FIGS. 14A-14B present schematic views of a working cannula in accordance with one or more embodiments.

FIG. 15 is a schematic view of a delivery device in accordance with one or more embodiments.

FIG. 16 is a schematic view of an anti-rotation driver in accordance with one or more embodiments.

FIG. 17 is a schematic view of a plunger in accordance with one or more embodiments.

FIGS. 18A-18B present schematic views of a screwdriver shaft for implant insertion in accordance with one or more embodiments.

FIG. 19 is a schematic view of a plunger in accordance with one or more embodiments.

FIG. 20 presents a schematic view of an implant having an integrated joint finder in accordance with one or more embodiments.

FIG. 21 presents a scanning electron microscope (SEM) image of a representative implant surface structure in accordance with one or more embodiments.

FIG. 22 presents an energy-dispersive X-ray spectroscopy (EDS) spectrum of a representative implant surface structure in accordance with one or more embodiments.

FIG. 23 summarizes surface roughness properties of a representative implant in accordance with one or more embodiments.

FIG. 24 summarizes physical properties of porous material of a representative implant in accordance with one or more embodiments.

### DETAILED DESCRIPTION

In accordance with one or more embodiments, orthopedic implants are disclosed. The implants may generally promote fusion between two or more pieces of bone to promote healing. The implants may transfix and stabilize two bones, e.g. bone fragments, while limiting their motion. In some embodiments, the implants may bridge a space or gap between the pieces of bone. Beneficially, the geometry of the implants may obviate the need for use of supplemental fixation and may limit anatomical disturbances. The geometry may also facilitate insertion while imparting sufficient mechanical strength. Other structural features of the implants, such as but not limited to void space, surface roughness and porosity, as disclosed herein may generally promote bone growth and fusion. In some non-limiting embodiments, the disclosed implants may find particular utility in orthopedic applications. In one specific embodiment, the implants may be used for sacroiliac (SI) joint fusion. In another specific embodiment, the implants may be used to fuse vertebrae of a subject.

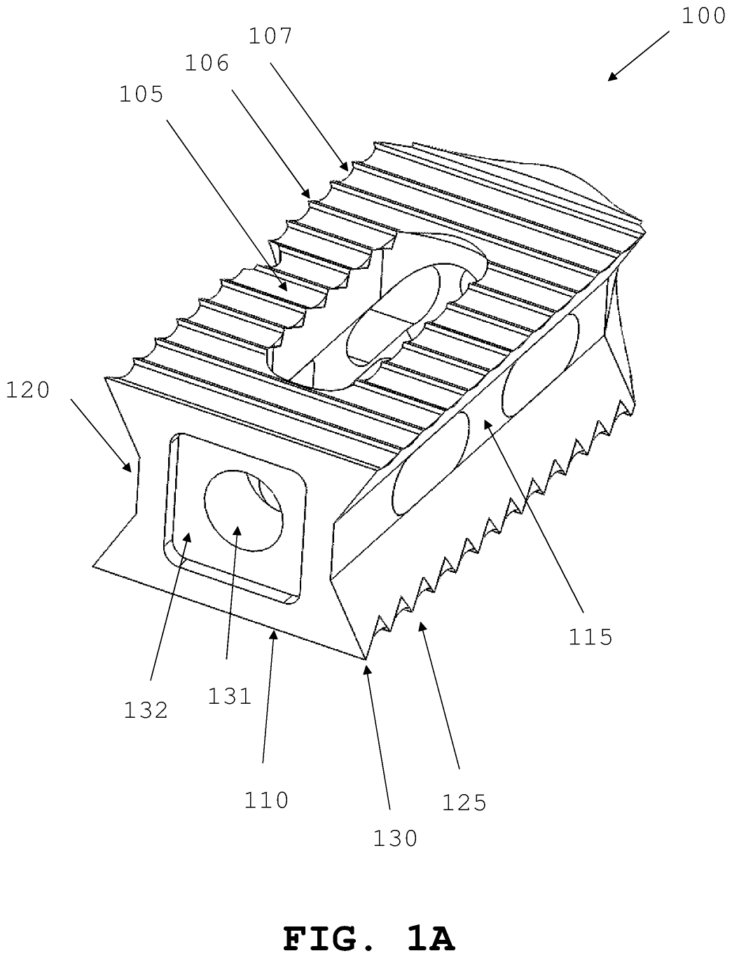

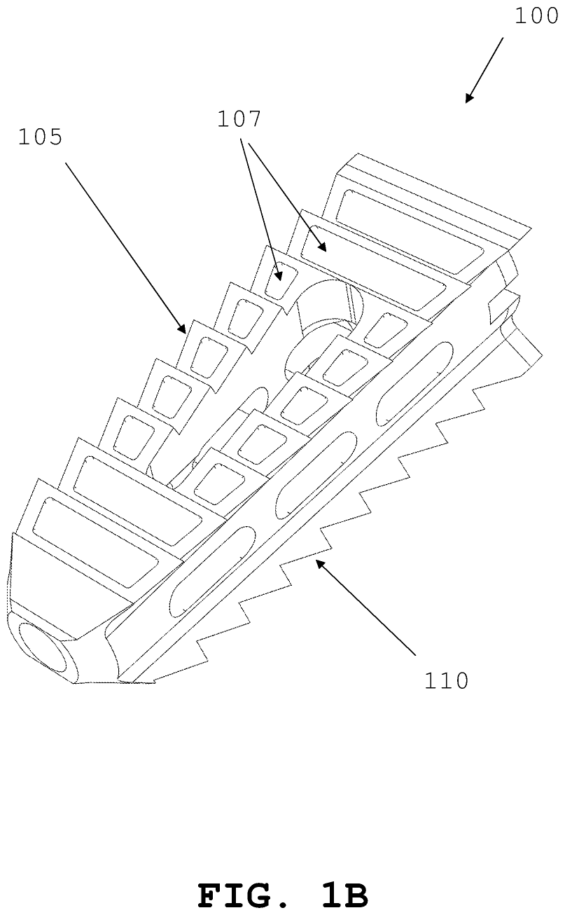

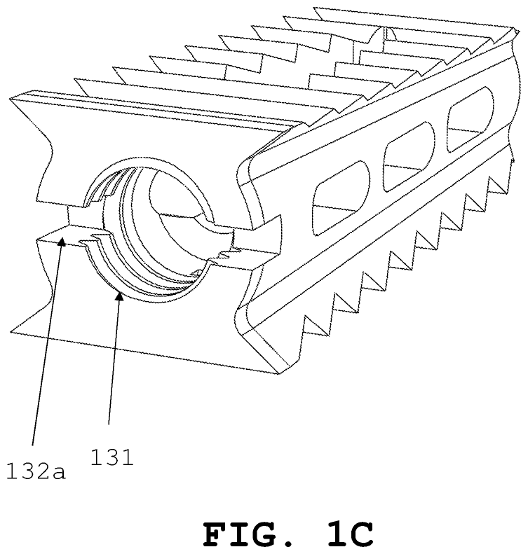

Looking first at FIG. 1A there is shown a novel transfixing wedge 100 for fixating two adjacent bones so as to eliminate relative motion between the two bones and generate fusion between them. Transfixing wedge 100 is manufactured from a titanium alloy such as Titanium-6Aluminum-4Vanadium; however, it may be manufactured from any other biocompatible metal, polymer, or autograft, allograft, or xenograft bone. The material, in conjunction with the wedge geometry as described herein, should generally be selected for and capable of imparting requisite mechanical properties for an intended application. Specifically, it may be manufactured from polyetherketoneketone (PEKK) or polyetheretherketone (PEEK) so as to make the implant show up as more radiolucent on an x-ray or fluoroscopy. Transfixing wedge 100 may be traditionally machined or may be manufactured using additive manufacturing methods. Without wishing to be bound to any particular theory, additive manufacturing techniques may enable unique geometries and surface features, both interior and exterior, as described herein for the wedge implant while meeting requisite mechanical properties. In a preferred but non-limiting embodiment, transfixing wedge 100 is additively manufactured from a titanium alloy so as to have a macro rough surface with a surface roughness (Ra) of approximately 0.020 to 0.060 mm, and preferably approximately 0.0430 mm and subsequently electrochemically treated to create an additional uniform nano-textured roughness that exhibits a “net-like” structure comprising thin nanometric sharp crests, with crest-to-crest nano-texture roughness measurements between 10 and 75 mm and preferably approximately 40 mm, and a surface oxide rich in calcium and phosphorous.

In an alternative preferred but non-limiting embodiment, transfixing wedge 100 is additively manufactured from polyetherketoneketone (PEKK) or polyetheretherketone (PEEK) so as to make the implant show up as more radiolucent on an x-ray or fluoroscopy. Specifically, when transfixing wedge 100 is manufactured from PEKK, the macro surface roughness of the implant (Ra) ranges from approximately 0.005 mm to 0.050 mm, and preferably approximately 0.026 mm. Furthermore, the implant surface has nanometer surface features that increase the surface area of the implant. The nanometer surface features range in size from 50 to 100 nm, and are preferably around 75 nm. The surface is highly hydrophobic, with a contact angle of approximately 108 degrees. This combination of surface roughness and surface energy may give the implant antibacterial properties.

Transfixing wedge 100 is elongated with a non-rectangular cross-section. The cross-section of the wedge implant is generally constructed and arranged to promote bone fixation upon introduction. The wedge implant may include one or more structural features in cross-section which interlocks with bone upon insertion to promote fixation. Various geometries are envisioned such that translation is generally prevented upon impact of the implant to stabilize the bones. In at least some non-limiting embodiments, such structural features may be referred to as interlocking features, e.g. dovetail features. The interlocking features may capture and/or otherwise engage bone upon implantation to promote fixation and prevent rotation. These structural features may generally be associated with side walls of the wedge implant as described further herein.

In one preferred but non-limiting embodiment, the wedge implant may have a cross-sectional shape defined by two opposed dovetails around a central axis that serves to transfix adjacent bones. The wedge implant and bones may form opposed dovetail joints in which tapered projections (tenons) in one element mate with corresponding notches, indents or recesses (mortises) in the other element for stability and fixation. Projections of the wedge implant may run along the length of the side walls and may be referred to as dovetail edges herein. Recesses of the wedge implant may run along the length of the side walls between the projections. The recesses may generally be defined by the contour of the projections and/or side walls. In some non-limiting embodiments, each side wall of the wedge implant may have a pair of projections defining a single recess. Other embodiments may include multiple pairs of projections on each side wall with each pair defining a recess.

Transfixing wedge 100 has a top surface 105 and a bottom surface 110. Surfaces 105 and 110 may be parallel or slope toward each other over the length of the implant with the two surfaces spaced closer together at the distal end. When surfaces 105 and 110 slope toward each other, insertion of the implant may be easier as well as act as a wedge to create distraction between the two bones. Top and bottom surfaces 105 and 110 may have teeth or ridges 106 on their surfaces to prevent implant migration. In accordance with one or more embodiments, top surface 105 may generally have a projection of the interlocking feature, e.g. dovetail feature, along each longitudinal edge. Likewise, bottom surface 110 may generally have a projection of the interlocking feature, e.g. dovetail feature along each longitudinal edge. These projections may be associated with side walls 115, 120 as discussed below.

Porous material 107 manufactured from the same material as the transfixing wedge (e.g., Titanium Alloy) may be placed between adjacent teeth to promote bone growth and fusion. Porous material 107 has a pore size ranging between 100 mm and 700 mm, and a pore size most preferably around 300-500 mm. Additionally, the surface roughness (Ra) of the porous material 107, is approximately 0.040 to 0.100 mm, and preferably approximately 0.0608 mm. The roughness of the porous material is greater than that of the surface roughness of the implant. This is purposeful, as it is beneficial for the porous material to have a greater roughness to promote early bone attachment and to ensure contact with the adjacent bone.

Alternatively, and now looking at FIG. 1B, the porous material 107 may be located in pockets on the top and bottom surfaces 105 and 110.

Looking back at FIG. 1A, transfixing wedge 100 has side walls 115 and 120 which are perpendicular to surfaces 105 and 110 and are inset from dovetail edges 125. FIG. 1C presents a different geometry for side walls 115 and 120 in which they are also inset but with a semi-circular contour which, in turn, define a corresponding contoured recess. Other geometries are envisioned and may be implemented depending on the intended application. Thus, the four corners 130 of the implant cross section extend beyond the side wedge walls 115 and 120. This creates a dovetail shaped implant. Side walls 115 and 120 may be parallel or may slope toward each other over the length of the implant with the two surfaces spaced closer together at the distal end.

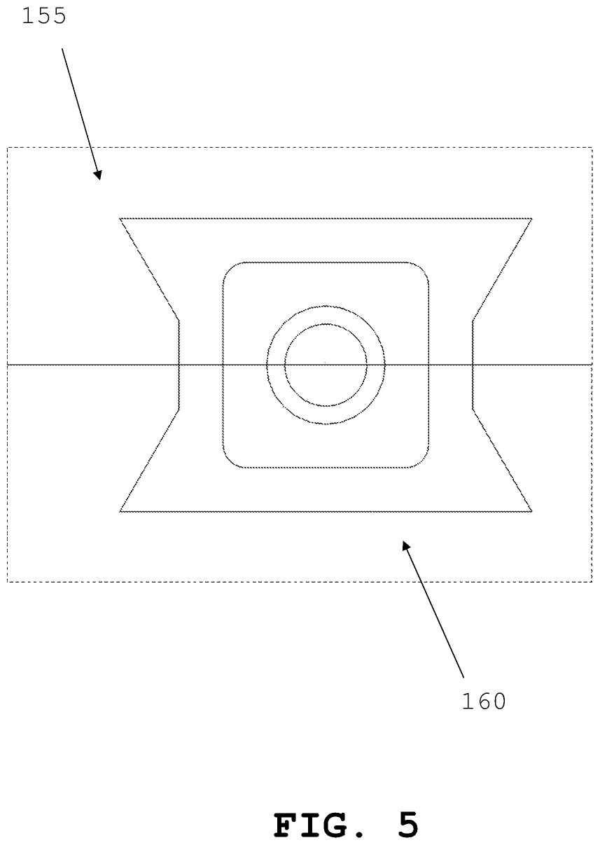

Side walls 115 and 120 each interlocks with both first and second bones upon insertion of the wedge implant in view of the cross-sectional geometry as described above with opposed dovetail features. Specifically, the projections and recess at a first side of the wedge implant interlock with first and second bones, and the projections and recess at a second side of the wedge implant interlock with the first and second bones. The top surface 105 is generally associated with one of the first and second bones, and the bottom surface 110 is generally associated with the other of the first and second bones as illustrate in FIG. 5.

In accordance with one or more embodiments, one or more dimensions of the wedge implant may vary depending on an intended application. In some embodiments, it may be desirable for first and second bones to be substantially in contact with one another or abutting when the wedge is implanted therebetween. In other embodiments, it may be desirable for the wedge implant to span or bridge a gap or space between first and second bones upon insertion. In some embodiments, the wedge implant may be sized such that a distance between top and bottom surfaces 105, 110 is selected in order to control the span of the wedge implant.

In other embodiments, it may be desirable for the wedge implant to distract and create space for the implant to span or bridge between first and second bones upon insertion, and for the surrounding intact soft tissue to resist the distraction and generate a compressive force between the first and second bones (i.e., ligamentotaxis or distraction arthrodesis).

In accordance with one or more embodiments, the extent of inset of side walls 115, 120 relative to the associated dovetail edges may vary. Adjusting an angle between a dovetail edge and an associated side wall may control the extent of the inset. In some non-limiting embodiments, said angle may range between 20 degrees and 70 degrees and preferably may be approximately 45 degrees. The angle may also vary from the proximal end to the distal end of the implant. A deeper inset may be associated with greater fixation, capture, engagement and/or interlock with bone. The extent of the inset, however, may influence strength, stability and other mechanical properties of the wedge implant and must be balanced. The extent of the inset may also influence the overall as well as interior geometry of the wedge implant as described herein.

Transfixing wedge 100 has an attachment feature 131 for attaching the wedge onto an inserter. Attachment feature 131 may be a threaded hole, press fit, bayonet fitting, or other appropriate attachment feature. Additionally, when attachment feature 131 is a thread, it may be beneficial to have an anti-rotation tool on the delivery device. This anti-rotation tool can interface with the implant via an internal recess 132, or slot 132a (FIG. 1C) on the end of the implant. Thus, it is possible to unscrew transfixing wedge 100 from the delivery device without the wedge rotating. Additionally, attachment feature 131 can be used to direct a flowable biologically active agent into and through the transfixing wedge.

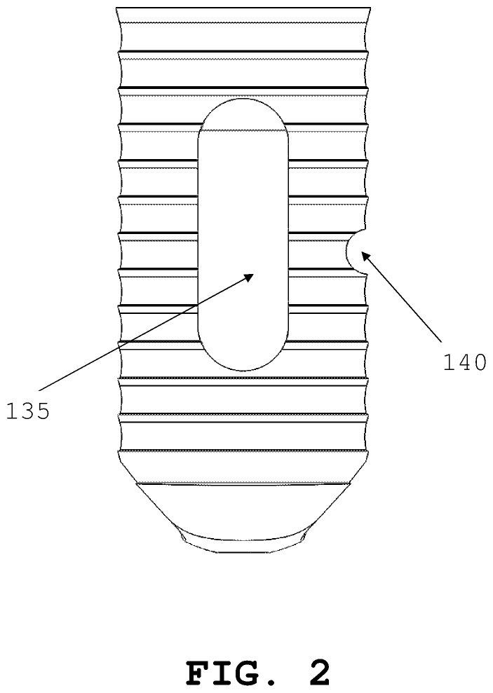

Looking now at FIG. 2 there is shown the top of transfixing wedge 100. The transfixing wedge has a central lumen 135 that extends from surface 105 to 110. Central lumen 135 may be an empty space or may be filled with porous material manufactured from the same material the transfixing wedge is (e.g., Titanium Alloy). The transfixing wedge may also have a visual indicator 140 to identify and differentiate side wall 115 and 120. The visual indicator may be a cutout, slot, bead, dent, bump or a marker (e.g., tantalum marker) that is visible under x-ray, fluoroscopy, or CT.

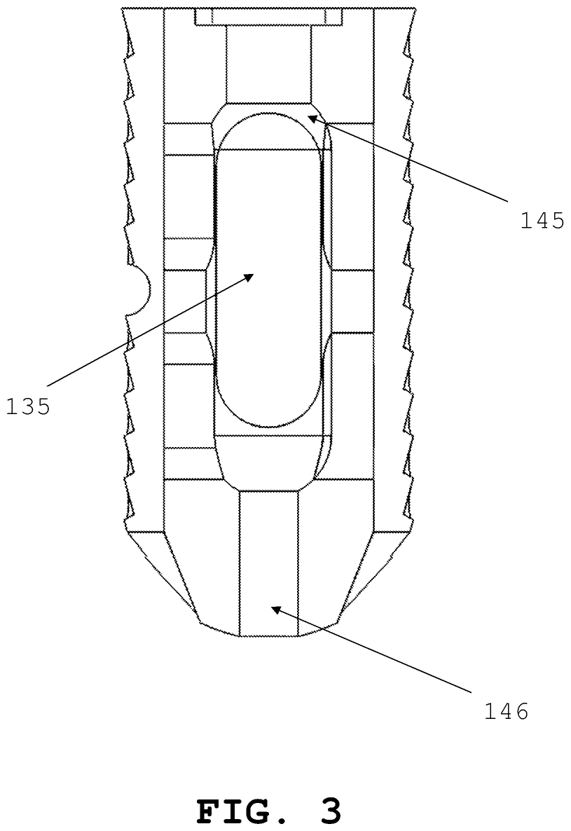

Looking now at FIG. 3, a cross-sectional view of the transfixing wedge 100 is shown. Central lumen 135 may extend within the body of the implant creating a reservoir for bone growth agents or providing additional surface area for bone to grown onto. Additionally, a central cannulation 146 may be present throughout the length of the implant and run along the central axis of the wedge. Central cannulation 146 allows the implant to be implanted over a k-wire.

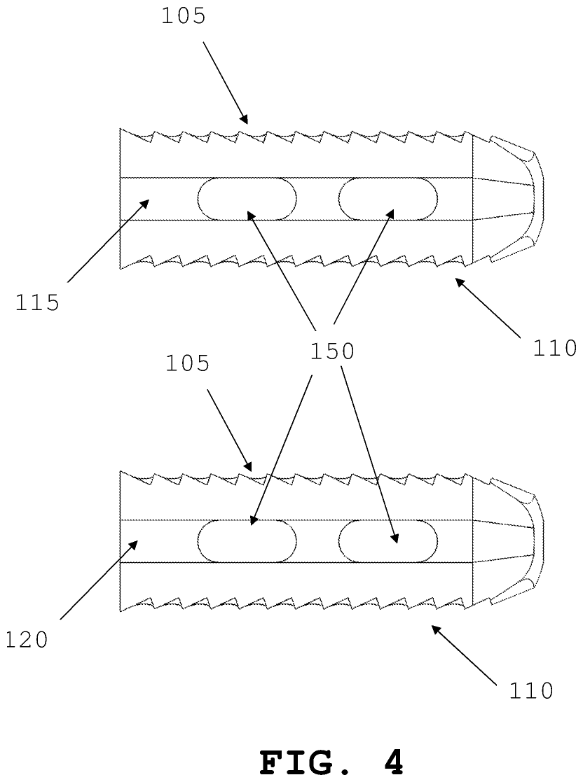

Looking now at FIG. 4, side views of the transfixing wedge 100 are shown. Side walls 115 and 120 may be the same or they may be different. The side walls may have one or more windows 150. The windows may be circular, oval, rectangular, polygonal, or other shaped. The windows may be open to the central lumen 135 or may be filled with a porous material manufactured from the same material the transfixing wedge is (e.g., Titanium Alloy). Thus, it is possible to direct the flow of a flowable biologically active agent. In one embodiment, windows 150 on side wall 115 are open, and windows 150 on side wall 120 are filled with a dense porous material, thus when a flowable biologically active agent is delivered under pressure to the transfixing wedge, the flowable biologically active agent will flow out of the windows on side wall 115 and not out of the windows on side wall 120. This allows for controlling the delivery of a flowable biologically active agent.

In some embodiments, the flowable biologically active agent may include bone growth promoting material. In some embodiments, the flowable biologically active agent may include therapeutic agents and/or pharmacological agents for release, including sustained release, into a surrounding tissue to treat, for example, pain, inflammation and degeneration. The agents may include pharmacological agents, such as, for example, antibiotics, pain medications, analgesics, anesthetics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), and combinations thereof. In some embodiments, the agent may include bone cement that enhances fixation of the wedge 100 with tissue. In some embodiments, the bone cement may include a poly (methyl methacrylate) (PMMA); methyl methacrylate (MMA); calcium phosphate; a resorbable polymer, such as, for example, PLA, PGA or combinations thereof; a resorbable polymer with allograft, such as, for example, particles or fibers of mineralized bone and/or combinations thereof.

Now looking at FIG. 5, novel transfixing wedge 100 is shown implanted into bone and preventing motion of bone fragment 155 and 160. The dovetail shape of the implant does not allow the two bones to be pulled apart. Pulling apart bone fragments 155 and 160 generally requires in excess of 150 N of force. Transfixing wedge 100 resists axial pullout (pullout along the central axis of the implant) similarly to a threaded screw. In accordance with one or more embodiments, the pullout-force of a representative implant may be greater than 600 N. A sufficient pullout-force is required for an implant that does not rely on supplemental fixation so that the implant does not back-out or experience expulsion while healing occurs. In an exemplary and non-limiting embodiment, bone fragment 155 is the ilium and bone fragment 160 is the sacrum. In this example the novel transfixing wedge 100 is used to perform a sacroiliac joint fusion.

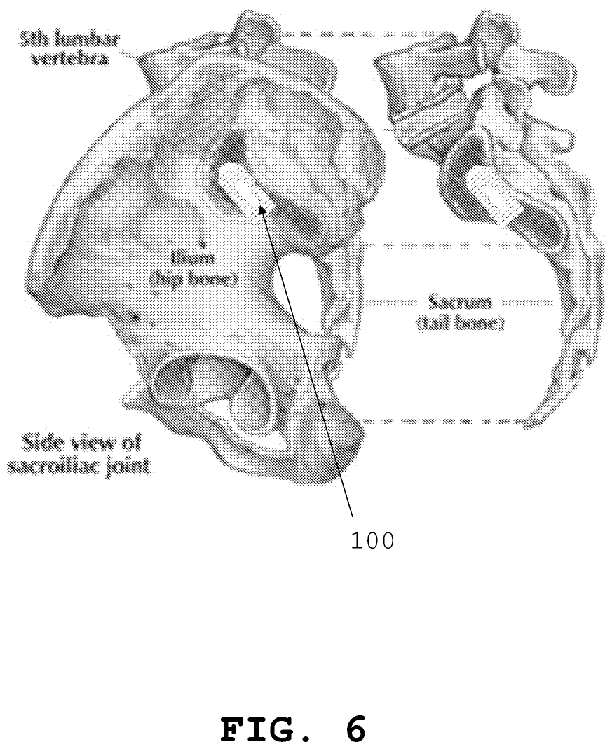

Now looking at FIG. 6, novel transfixing wedge 100 is shown implanted within the sacroiliac (SI) joint.

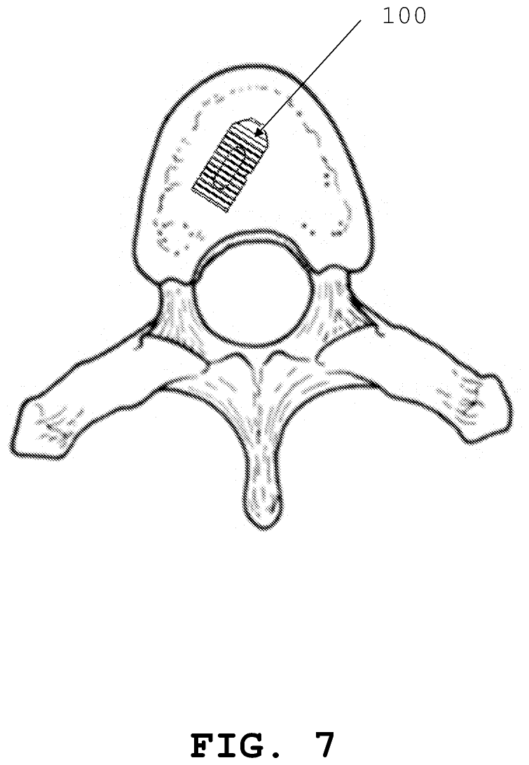

Now looking at FIG. 7, novel transfixing wedge 100 is shown implanted between two adjacent vertebrae.

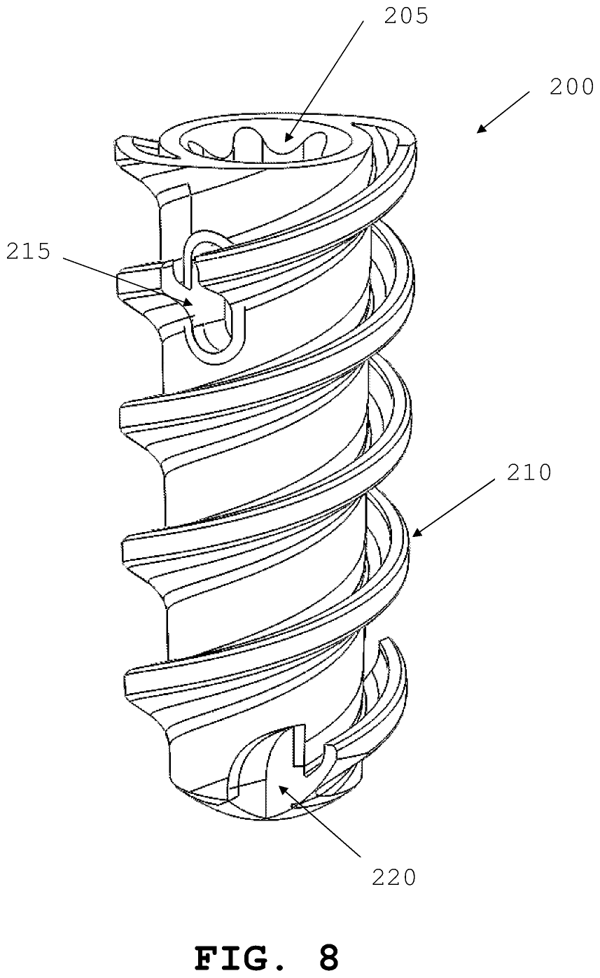

Looking at FIG. 8 there is shown a novel transfixing screw 200 for fixating two adjacent bones so as to eliminate relative motion between the two bones and generate fusion between them. Transfixing screw 200 is manufactured from a titanium alloy such as Titanium-6Aluminum-4Vanadium; however, it may be manufactured from any other biocompatible metal, polymer, or autograft, allograft, or xenograft bone. Specifically, it may be manufactured from polyetherketoneketone (PEKK) or polyetheretherketone (PEEK) so as to make the implant show up as more radiolucent on an x-ray or fluoroscopy. Transfixing screw 200 may be traditionally machined or may be manufactured using additive manufacturing methods. In a preferred embodiment, transfixing wedge 200 is additively manufactured from a titanium alloy so as to have a macro rough surface with a surface roughness (Ra) of approximately 0.0430 mm and subsequently electrochemically treated to create an additional uniform nano-textured roughness that exhibits a “net-like” structure comprising thin nanometric sharp crests, with crest-to-crest nano-texture roughness measurements between 10 and 75 mm and a surface oxide rich in calcium and phosphorous.

Now looking at FIG. 8, transfixing screw 200 has a drive feature 205 for inserting the screw into the bones. The drive feature may be any feature known in the art including but not limited to torx, hex, square, philips, hexalobe, slot, etc. The screw has a thread 210 wrapped helically around a central longitudinal axis. The thread may have a single start, or multiple starts so as to reduce the number of rotations needed for inserting the screw. Transfixing screw 200 may also have one or more windows 215 projecting radially from a central plane of the screw. These windows can allow a flowable biologically active agent to pass through the window and into and around the bone to be fused. Additionally, transfixing screw 200 may have features 220 on the tip of the screw for helping the thread cut into the bone and ease thread insertion.

In some embodiments, the flowable biologically active agent may include bone growth promoting material. In some embodiments, the flowable biologically active agent may include therapeutic agents and/or pharmacological agents for release, including sustained release, into a surrounding tissue to treat, for example, pain, inflammation and degeneration. The agents may include pharmacological agents, such as, for example, antibiotics, pain medications, analgesics, anesthetics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), and combinations thereof. In some embodiments, the agent may include bone cement that enhances fixation of the cage 1 with tissue. In some embodiments, the bone cement may include a poly (methyl methacrylate) (PMMA); methyl methacrylate (MMA); calcium phosphate; a resorbable polymer, such as, for example, PLA, PGA or combinations thereof; a resorbable polymer with allograft, such as, for example, particles or fibers of mineralized bone and/or combinations thereof.

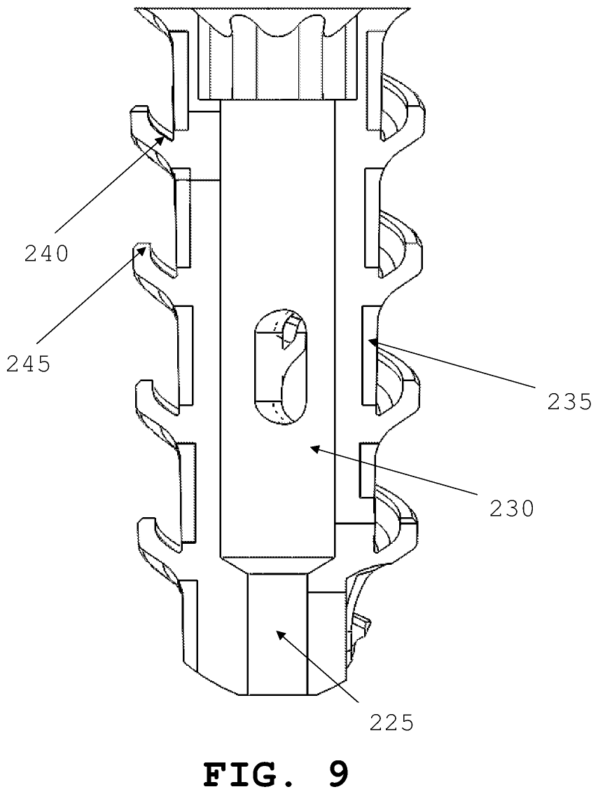

Now looking at FIG. 9, a cross-section of the transfixing screw is shown. The transfixing screw can have a central cannulation 225 that runs the length of the screw. The central cannulation allows the screw to be implanted over a k-wire, in order to aid in the positioning of the screw. Additionally, the cannulation may have a central lumen 230. Central lumen 230 may be empty space or may be filled with porous material manufactured from the same material the transfixing screw is (e.g., Titanium Alloy). Central lumen 230 may create a reservoir for bone growth agents or providing additional surface area for bone to grown onto. Additionally, within the thread valley, a porous structure 235 manufactured from the same material as the transfixing screw (e.g., Titanium alloy) can be helically wrapped around the screw. This porous material is designed to promote bone on growth and in growth. Porous structure 235 has a pore size ranging between 100 mm and 700 mm, and a pore size most preferably around 300-500 mm. Additionally, the surface roughness (Ra) of the porous structure 235, is approximately 0.0608 mm. The roughness of the porous material is greater than that of the surface roughness of the implant. This is purposeful, as it is beneficial for the porous material to have a greater roughness to promote early bone attachment and to ensure contact with the adjacent bone.

Thread 210 has an upward and outwardly sloping surface 240 that terminates at a more vertical surface 245. Surface 245 is positioned at an angle relative to 240 so as to create a hook that can capture bone and resists pull apart.

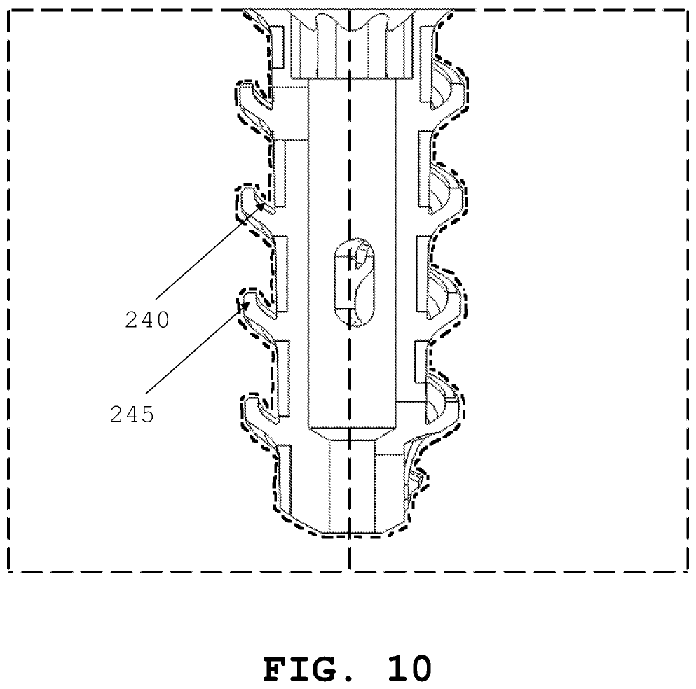

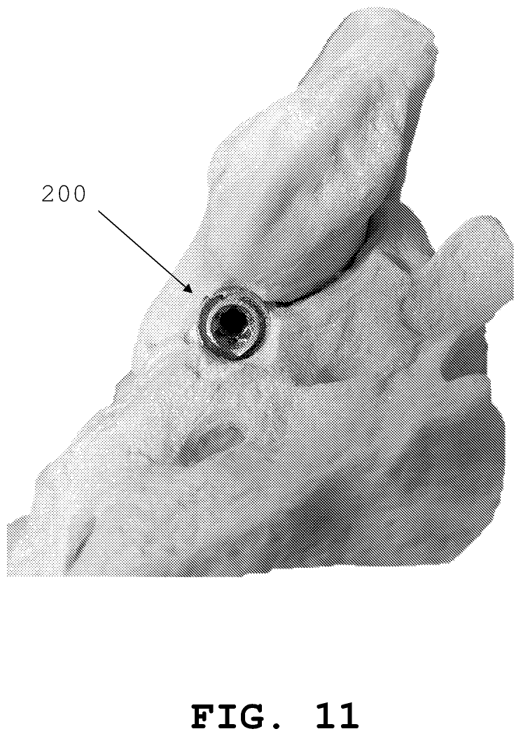

Now, looking at FIG. 10, transfixing screw 200 is shown transfixing two adjacent bones. The combined profile of surfaces 240 and 245 cuts into the adjacent tissue and inhibits motion away from the central axis of the screw. FIG. 11 shows transfixing screw 200 transfixing the sacroiliac (SI) joint.

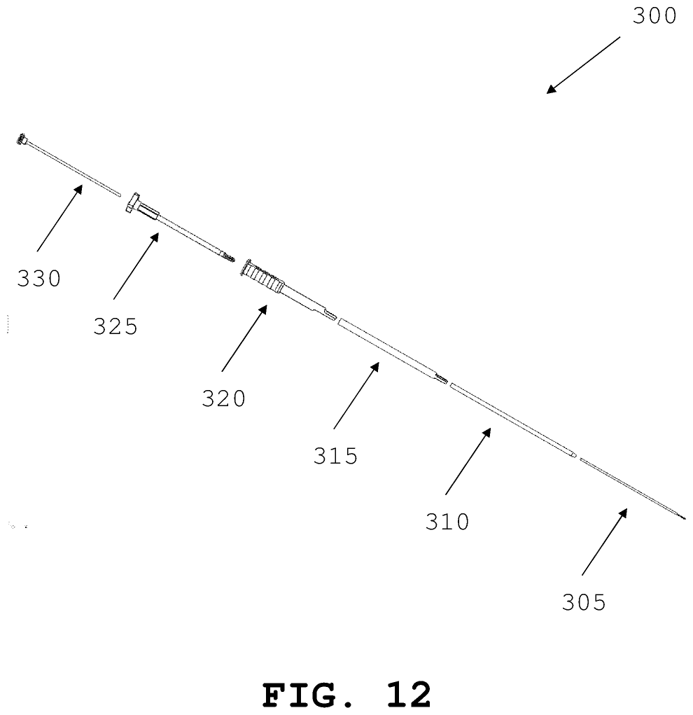

Now looking at FIG. 12, the components 300 of a transfixing implant implantation kit are shown. The transfixing kit may include any number of but not limited to the following components: a k-wire, 305, an introducer, 310, a joint finder, 315, a working cannula 320, and implant delivery device, 325, and a biologically active agent delivery device, 330. Depending on the implant design and the patient's anatomy, some of these components may not be needed.

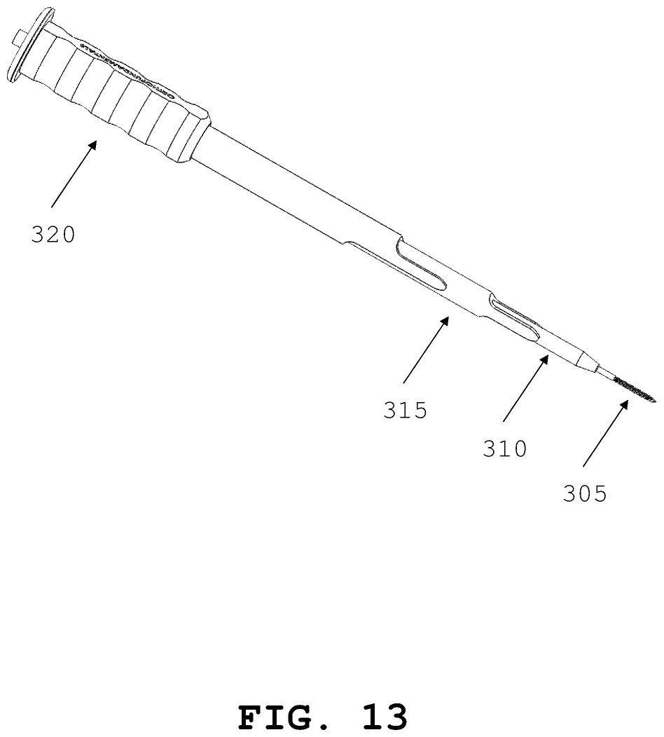

Looking now at FIG. 13, the components of the device needed to control the placement of the implant are shown. K-wire 305 is first inserted to identify the anatomy and ensure the desired trajectory is achieved. The k-wire may be blunt, threaded, or have a trocar style point. The location of the k-wire can be observed using intra-operative imaging. Over the k-wire an introducer, 310 can be used to create a pathway to the bone surface. Depending on the patient and the anatomy, the introducer 310, may not be used and instead the joint finder, 315 can slide directly over the k-wire and into the joint to be transfixed. If the introducer 310 is used, the joint finder can slide directly over it. The joint finder may be designed with two “fingers” that are sized to enter the joint space. They may be sized to require impaction to enter the joint space, and they may create distraction of the joint. Finally, the working cannula 320, is slid over the joint finder. The working cannula also has two “fingers” that are sized to enter the joint space. They may be sized to require impaction to enter the joint space, and they may create distraction of the joint.

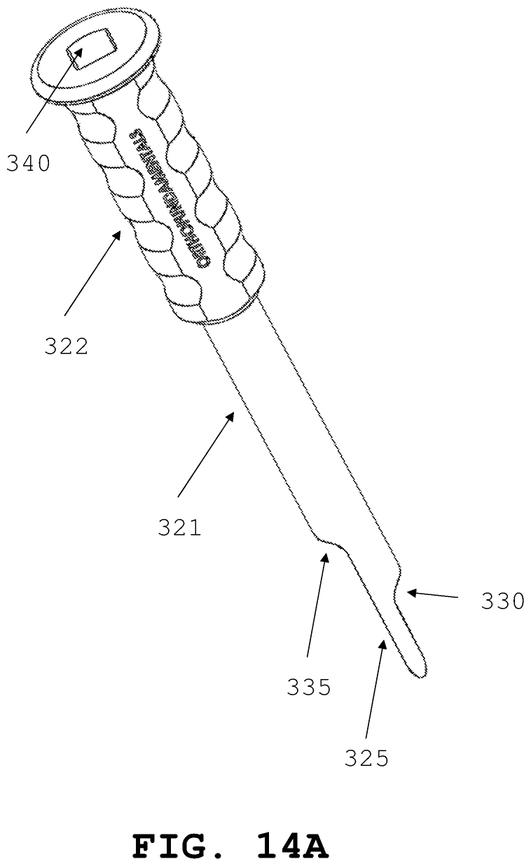

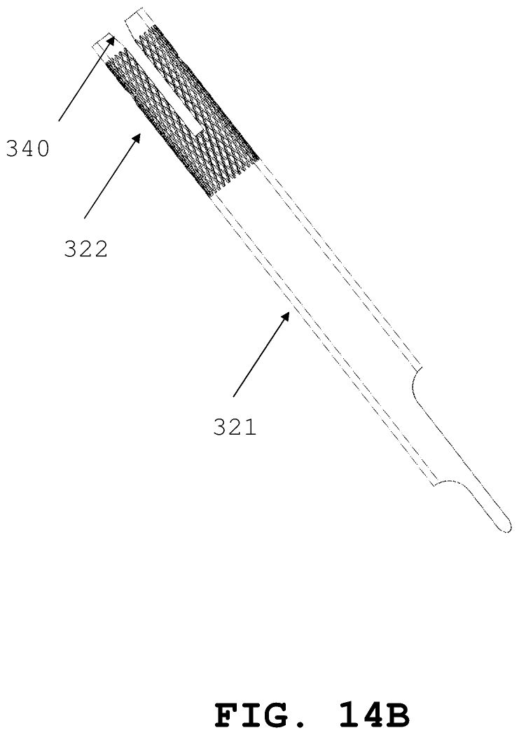

Now looking at FIG. 14A, the working cannula 320 is shown in more detail. The working cannula has a cylindrical body 321 and a handle 322. Handle 322 may be integral to cylindrical body 321 (FIG. 14B). The cylindrical body has fingers, 325 of the working cannula create two recesses in the cannula. Recess 330 is sized to abut the sacrum and is shorter. Recess 335 is longer and is sized to abut the pelvis. The top of handle 322 has a recess 340 sized to allow the passage of the delivery device. Recess 340 may not be symmetrical to create a directionality for the insertion of the delivery device. Recess 340 may be a slot and may not be symmetrical (FIG. 14B).

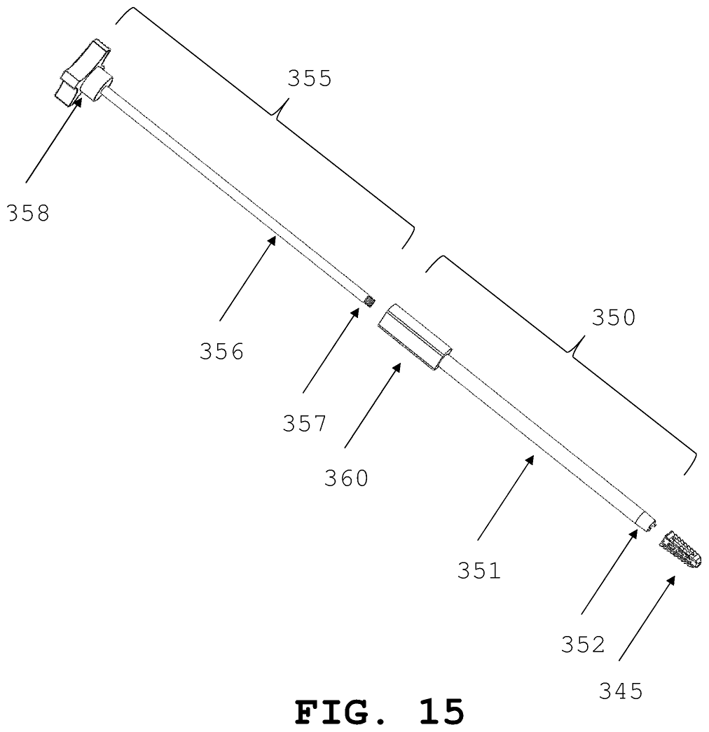

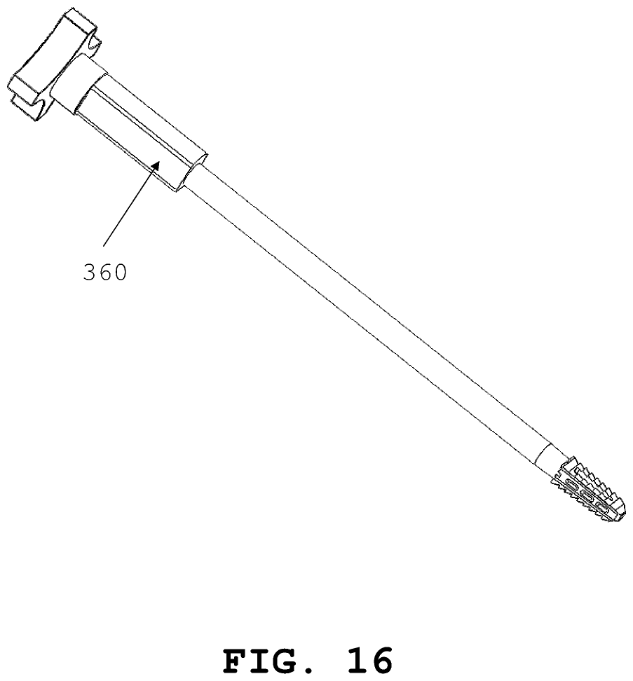

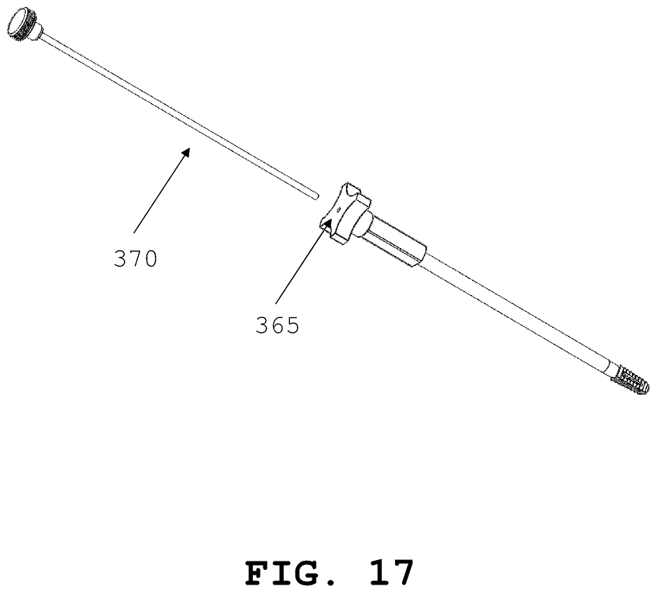

Looking now at FIG. 15, a representative delivery device is shown for the wedge style implant. The delivery system is composed of an inner threaded driver 355 and an anti-rotation driver 350. The threaded driver 355 is made up of a tube 356, with a threaded end 357. On the opposite side a handle 358 is installed. The handle may be cannulated. The anti-rotation driver 350 is made up of a tube 351 with an implant mating feature 352 on one end. On the other end a handle 360 is installed. The inner threaded driver 355 can pass through outer anti-rotation driver 350. Implant 345 can be threaded onto the inner threaded driver 355, and keyed onto anti-rotation driver 350 as shown in FIG. 16. The anti-rotation driver has a handle 360 that is asymmetrical and is designed to mate with recess 340 on the working cannula. Thus, the surgeon can know the orientation of the implant when it is being inserted. This may be important if the implant has passageways to allow for implantation of biologically active agents. This feature allows the surgeon to control the direction the biologically active agent flows out of the implant. Biologically active agents can be inserted into and through the implant following implantation. Looking now at FIG. 17, it is possible to transfer a biologically active agent into the inner threaded driver via an opening 365 in the driver handle. A plunger 370, can then push the biologically active agent down the threaded inserter and into the implant.

It should be appreciated that an implant of the style described in FIGS. 1-7 may not need a pilot hole to first be drilled prior to insertion of the implant. The implant can be designed to self-broach and decorticate while it is impacted into the joint space. In particularly hard bone it may be required to first drill a pilot hole.

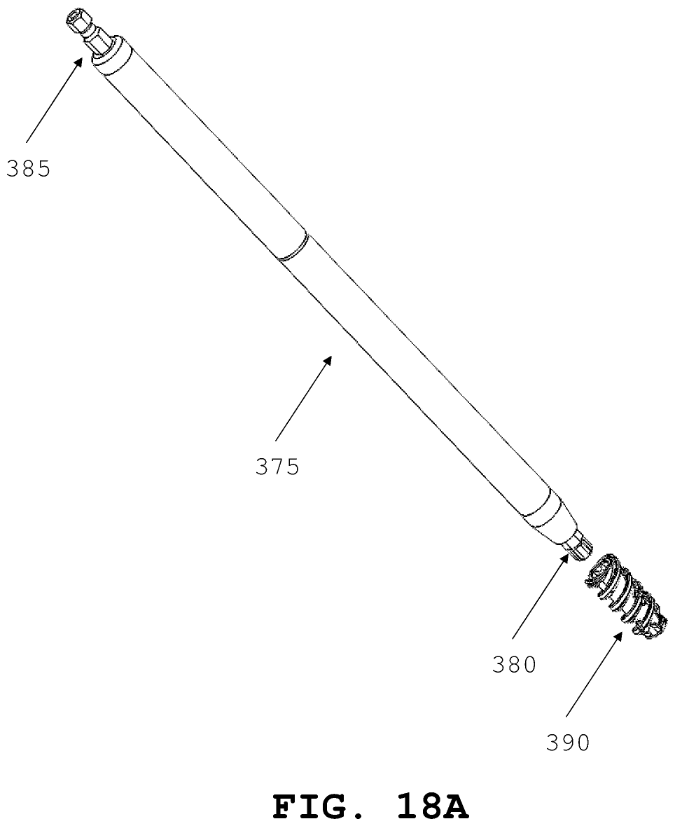

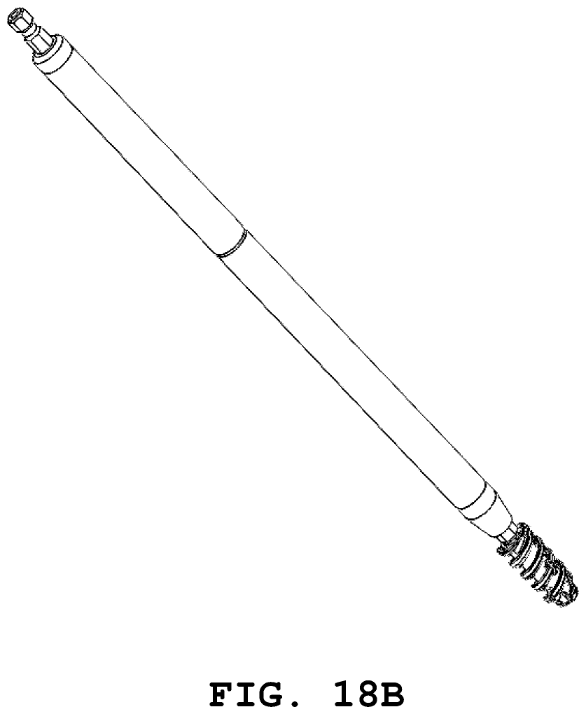

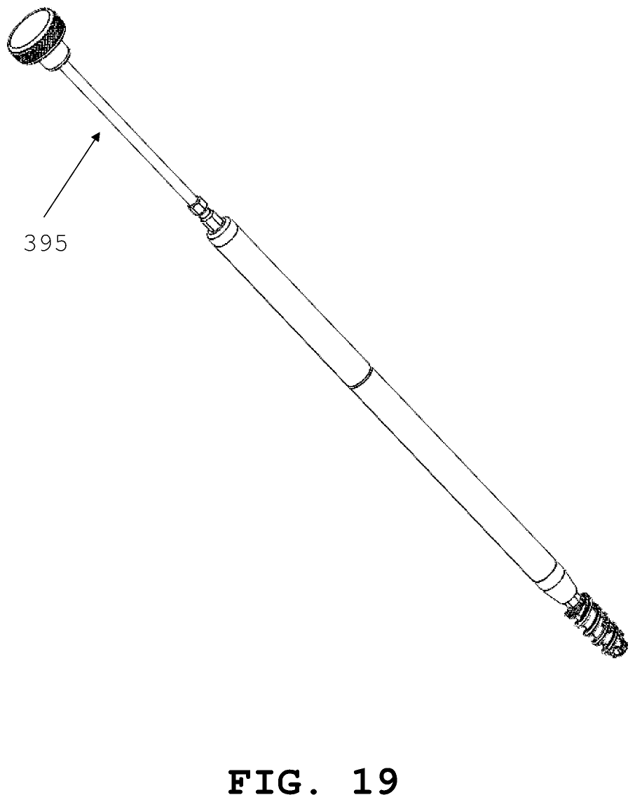

Alternatively, for a screw-based implant, and now looking at FIG. 18A, a screwdriver shaft 375 can be used to insert the implant 390. Screwdriver shaft 375 has a screw engagement feature 380 on one end. The feature may be a torx, hexalobe, philips, slot, or other screw engagement feature known in the art. On the other side, a driver engagement feature 385 is located. This allows the screwdriver shaft to connect to a handle or drill to generate the torque needed to insert or remove the screw. FIG. 18B shows the screw implant on the screwdriver shaft. Finally, and now looking at FIG. 19, a plunger 395 can be used to insert a biologically active agent into the implant.

It should be appreciated that an implant of the style described in FIGS. 8-11 may not need a pilot hole to first be drilled prior to insertion of the implant. The implant can be designed to self-drill and decorticate while it is screwed into the joint space. In particularly hard bone it may be required to first drill a pilot hole.

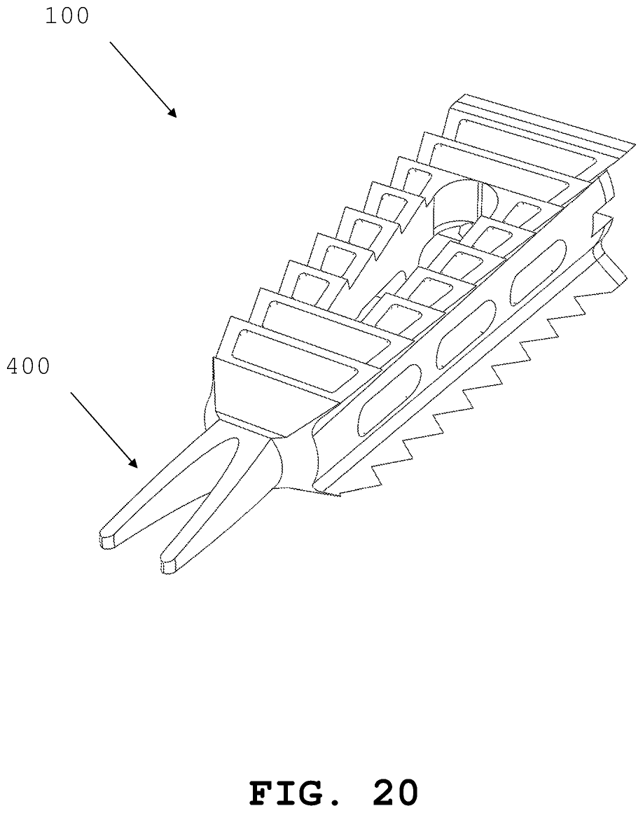

In an alternative embodiment, now looking at FIG. 20, novel transfixing wedge 100 has an integrated joint finder 400. This allows the implant to be inserted percutaneously, with no need for a joint finder, and in some cases without the need for a working cannula. The surgeon can use the integrated joint finder to locate the joint, and then impact the implant directly into the joint.

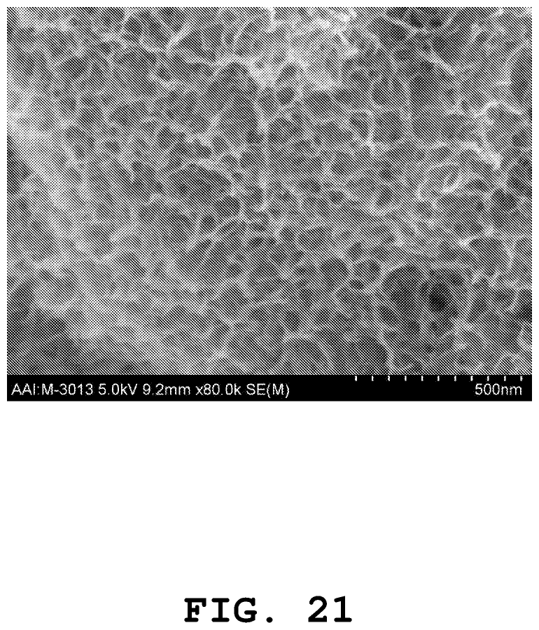

Looking now at FIG. 21, the electrochemical processing that may be performed to the implants creates a uniform surface covered by a thin nano-textured roughness that exhibited a “net-like” structure comprising thin nanometric sharp crests. The crest-to-crest nano-texture roughness measures between 10 and 75 nm in connection with a non-limiting representative implant. The nano-rough surface is on all surfaces of the implant, both on the outer surfaces, and within the porous materials.

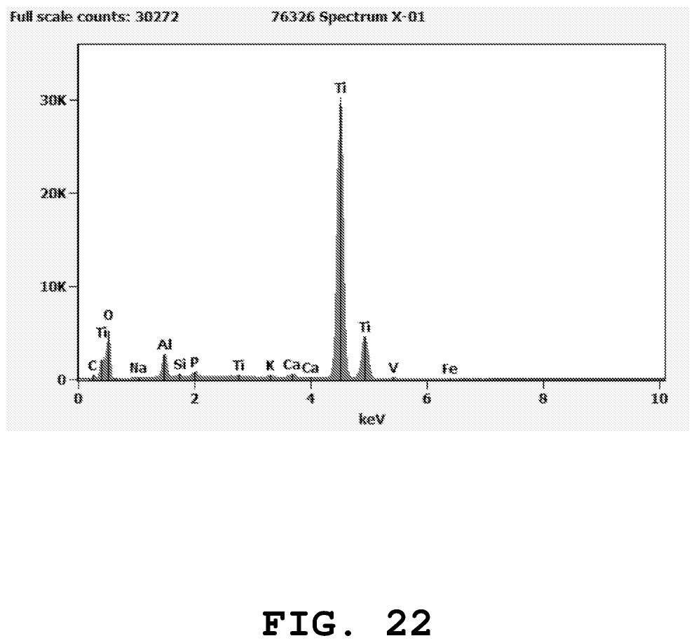

Looking now at FIG. 22, an EDS spectrum of the surface of the non-limiting representative implant referenced above shows the oxide layer containing calcium and phosphorous. Unlike traditional methods for coating implants with calcium and phosphorous (i.e., plasma spray) the calcium and phosphorous in the oxide layer of the hereby disclosed implant is chemically bound to the implant and is not easily removed while inserting the screw. Traditional coatings on an implant can easily be removed by the stresses of inserting the implant. Additionally, traditional methods for adding biologically active agents (calcium and phosphorous) to the surface of implants is a line-of-sight process. Thus, the biologically active agent will coat the outer surface of the implant, but not coat the inner surfaces or the porous material. The oxide layer of the implant in the present invention has an oxide layer on all surfaces (external and internal) of the implant that has calcium and phosphorous within the oxide.

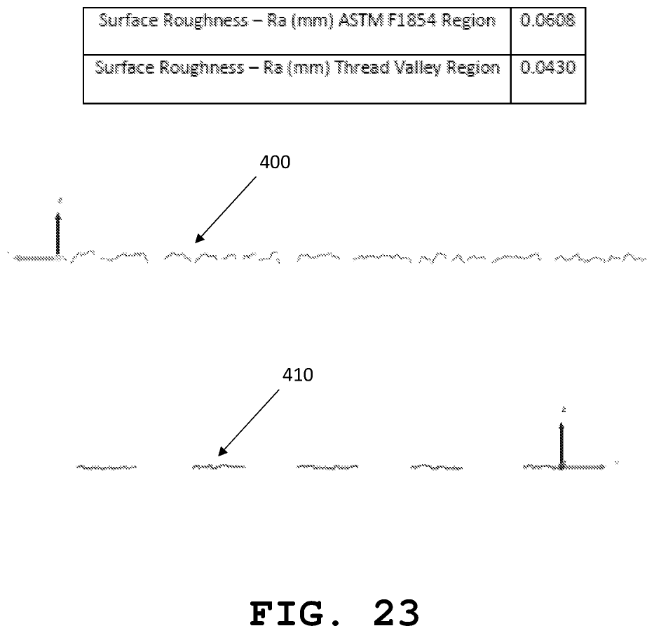

Looking now at FIG. 23, surface roughness properties are provided of the surface of the porous material 107 and 235, and of the implant surface in connection with a non-limiting representative implant. It should be appreciated that the surface roughness (Ra) of the porous material, 107 and 235, is approximately 0.0608 mm, while the surface roughness of the non-porous implant surface is 0.0430 mm. Thus, the roughness of the porous material is greater than that of the surface roughness of the non-porous material. This is purposefully, as it is beneficial for the porous material to have a greater roughness to promote early bone attachment and to ensure contact with the drilled hole.

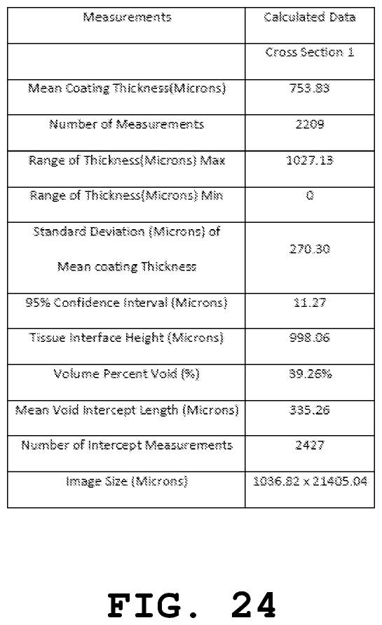

Looking now at FIG. 24, physical properties of the porous material 107 and 235 of a non-limiting representative implant are provided. The mean thickness of the porous material is approximately 753 mm, the tissue interface height is approximately 998 mm, and the mean void intercept length (pore size) is approximately 335 mm.

In accordance with one or more embodiments, a method of transfixing two adjacent bones is disclosed. The method may generally involve providing an implant as described herein. In some embodiments, no supplemental fixation is required. In at least some embodiments, no pilot hole is required.

In accordance with one or more embodiments, the implants disclosed herein may be used to transfix bones throughout the body of a subject. In some embodiments, the bones to be transfixed may be associated with an orthopedic application. In some non-limiting embodiments, the bones to be transfixed may be vertebrae. In at least some embodiments, the bones to be transfixed pertain to the sacroiliac (SI) joint of the subject.

In accordance with one or more embodiments, a method of stabilizing and fusing the sacroiliac joint without the use of a supplemental fixation is disclosed. The method may involve providing an implant as described herein.

In accordance with one or more embodiments, a method of fusing the sacroiliac joint without the use of a rotary cutting instrument or other abrading device is disclosed. The method may involve providing an implant as described herein.

In accordance with one or more embodiments, a repaired bone is disclosed. The repaired bone may generally involve an implant as described herein positioned between two adjacent bone fragments.

In accordance with one or more embodiments, a stabilized joint is disclosed. The stabilized joint may generally involve an implant as described herein positioned between two adjacent bones. In some embodiments, the two adjacent bones are in close proximity. In other embodiments, the implant bridges or spans a space or gap between the two adjacent bones. The space or gap may be less than a millimeter to approximately 5 mm. Preferably the space or gap is approximately 3 mm.

In accordance with one or more embodiments, a method of facilitating the transfixing of two adjacent bones is disclosed. The method may generally involve providing an implant as described herein. Instructions for use of an implant as described herein may also be provided.

In accordance with one or more embodiments, a kit is disclosed. The kit may generally include an implant as described herein. Other components for clinical use as described herein may also be included in the kit.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. An apparatus for securing a first bone fragment to a second bone fragment, said apparatus comprising:
a fusion device, said fusion device comprising:
an implant having a first face and a second face,
said first and second faces having a first and second edge,
a first side wall, and a second side wall wherein said first side wall and second side wall are perpendicular to said first face and second face, and wherein said first side wall and second sidewall are positioned medial to the edges of said first and second face so as to create an interlocking feature, e.g. a dovetail cross-section.

2. An implant according to claim 1 wherein said implant has a central lumen that spans the length of the implant.

3. An implant according to claim 2 wherein said implant has windows connecting the outside surface of said first side wall to the central lumen.

4. An implant according to claim 2 wherein said implant has windows connecting the outside surface of said first side wall to the central lumen, and windows connecting the outside surface of said second side wall to the central lumen that is filled with a porous material.

5. An implant according to claim 1 wherein said proximal end of said implant includes a feature for attachment to a delivery device.

6. An implant according to claim 1 wherein said proximal end of said implant includes a feature for attachment to a delivery device, and a separate feature for limiting rotation of said implant while removing said delivery device.

7. An implant according to claim 2 wherein said implant has windows connecting the outside surface of said first side wall to the central lumen, and windows connecting the outside surface of said second side wall to the central lumen that is filled with a porous material and wherein when a flowable biologically active agent is inserted under pressure into said proximal feature for attachment, said flowable biologically active agent flows from the attachment feature and out the first sidewall windows without flowing out of said second side wall windows.

8. An implant according to claim 1 wherein said first and second faces have a central window.

9. An implant according to claim 8 wherein said central window is filled with a porous structure.

10. An implant according to claim 9 wherein the pore size of the porous structure is between 100 and 1200 mm.

11. An implant according to claim 1 that has teeth on said first and second face to prevent implant migration.

12. An implant according to claim 11 wherein porous material is placed between adjacent teeth.

13. An implant according to claim 12 wherein the pore size of the porous material is between 100 and 600 mm.

14. An implant according to claim 1 wherein said implant's surface has a nano-roughness surface feature size of between 10 and 75 nm and the surface has a calcium and phosphorous present.

15. An apparatus for securing a first bone fragment to a second bone fragment, said apparatus comprising:
a fusion device, said fusion device comprising:
a cylindrical implant having a proximal and distal surface, a central axis, and a thread helically wound around said central axis, bridging said proximal and distal surfaces,
wherein said thread has an upwardly and outwardly sloping surface to capture bone during insertion and inhibit bone separation.

16. An implant according to claim 15 that has a hexalobe drive feature on said proximal surface.

17. An implant according to claim 15 that has a central cannulation.

18. An implant according to claim 15 that has a central lumen.

19. An implant according to claim 15 that has radially extending windows from said central lumen to the outer surface of said screw.

20. An implant according to claim 15 that has a porous material helically wound around said screws thread valley.

21. Porous material according to claim 20 that has a pore size of 100 to 600 mm.

22. An implant according to claim 15 wherein said implant's surface has a nano-roughness surface feature size of between 10 and 75 nm and the surface has a calcium and phosphorous present.

23. A method for transfixing two adjacent bones, said method comprising:
providing an implant, said implant comprising:
an implant having a first face and a second face,
said first and second faces having a first and second edge,
a first side wall, and a second side wall wherein said first side wall and second side wall are perpendicular to said first face and second face, and wherein said first side wall and second sidewall are positioned medial to the edges of said first and second face so as to create an interlocking feature, e.g. a dovetail cross-section.

24. A method according to claim 23 wherein said implant has a central lumen that spans the length of the implant.

25. A method according to claim 24 wherein said implant has windows connecting the outside surface of said first side wall to the central lumen.

26. A method according to claim 24 wherein said implant has windows connecting the outside surface of said first side wall to the central lumen, and windows connecting the outside surface of said second side wall to the central lumen that is filled with a porous material.

27. A method according to claim 23 wherein said proximal end of said implant includes a feature for attachment to a delivery device.

28. A method according to claim 23 wherein said proximal end of said implant includes a feature for attachment to a delivery device, and a separate feature for limiting rotation of said implant while removing said delivery device.

29. A method according to claim 24 wherein said implant has windows connecting the outside surface of said first side wall to the central lumen, and windows connecting the outside surface of said second side wall to the central lumen that is filled with a porous material and wherein when a flowable biologically active agent is inserted under pressure into said proximal feature for attachment, said flowable biologically active agent flows from the attachment feature and out the first sidewall windows without flowing out of said second side wall windows.

30. A method according to claim 23 wherein said first and second faces have a central window.

31. A method according to claim 30 wherein said central window is filled with a porous structure.

32. A method according to claim 31 wherein the pore size of the porous structure is between 100 and 1200 mm.

33. A method according to claim 23 that has teeth on said first and second face to prevent implant migration.

34. A method according to claim 33 wherein porous material is placed between adjacent teeth.

35. A method according to claim 34 wherein the pore size of the porous material is between 100and 600 mm.

36. A method according to claim 23 wherein said implant's surface has a nano-roughness surface feature size of between 10 and 75 nm and the surface has a calcium and phosphorous present.

37. A method of transfixing two adjacent bones, said method comprising:
providing an implant, said implant comprising:
a cylindrical implant having a proximal and distal surface, a central axis, and a thread helically wound around said central axis, bridging said proximal and distal surfaces,
wherein said thread has an upwardly and outwardly sloping surface to capture bone during insertion and inhibit bone separation.

38. A method according to claim 37 that has a hexalobe drive feature on said proximal surface.

39. A method according to claim 37 that has a central cannulation.

40. A method according to claim 37 that has a central lumen.

41. A method according to claim 37 that has radially extending windows from said central lumen to the outer surface of said screw.

42. A method according to claim 37 that has a porous material helically wound around said screws thread valley.

43. A method according to claim 42 that has a pore size of 100 to 600 mm.

44. A method according to claim 37 wherein said implant's surface has a nano-roughness surface feature size of between 10 and 75 nm and the surface has a calcium and phosphorous present.

45. An apparatus according to claim 15 wherein said thread has an upwardly and outwardly sloping surface to capture bone during insertion, said thread created to optimize bone capture whereby the angle of the termination point of the thread runs between 30 degrees and minus 30 degrees where 0 degrees represents the vertical direction in line with the central axis of the screw.

46. A method according to claim 37 wherein said thread has an upwardly and outwardly sloping surface to capture bone during insertion and inhibit bone separation, Said thread is formed at an angle ranging between 30 degrees and minus 30 degrees relative to the central axis of the screw to capture bone during insertion of the screw and wherein rotation of the screw during insertion drives the screw forward along the path whereby each consecutive rotation captures bone between the outer surface of the screw and the inner surface of the screw thread.

47. A method of fusing the sacroiliac joint without the use of a rotary cutting instrument or other abrading device, the method comprising the steps of:
locating an insertion point in the sacroiliac joint, the insertion point defining an intended location for the implant;
introducing a working cannula into the insertion point, the working cannula having an insertion end and a working end, the insertion end of the working cannula further comprising a first iliac contour, a first sacral contour, and insertion fingers; and
inserting an implant that transfixes the sacrum and the ilium.

48. A method according to claim 47, wherein the implant broaches its own opening while it is inserted via impaction.

49. A method according to claim 47, wherein the implant broaches its own opening while it is inserted via threading.

50. A method according to any of the preceding claims, further comprising securing the implant between first and second pieces of bone.

51. A method according to claim 50, wherein the method does not require supplemental fixation.

52. An orthopedic implant kit, comprising the apparatus of any of the preceding claims.

53. The kit of claim 52, further comprising one or more components selected from the group consisting of: an implant receiver, an introducer, a joint finder, a delivery device, a shaft, an anti-rotation tool, an indicator, a working cannula, a dilator, a k-wire, a drill, a screw driver, an impactor and/or a plunger.

54. The kit of claim 52, further comprising a source of a flowable biologically active agent.

55. The kit of claim 52, further comprising a source of a bone graft material, a bone cement and/or a source of a therapeutic agent.

56. A method of facilitating bone fusion, comprising:
providing the apparatus or kit of any of the preceding claims.

57. The method of claim 56, further comprising providing instructions for clinical use.

58. A repaired bone, comprising:
a first bone fragment;
a second bone fragment; and
an apparatus of any of the preceding claims interlocked between the first and second bone fragments.

59. The repaired bone of claim 58, wherein the apparatus bridges a space between the first and second bond fragments.

60. A reinforced joint, comprising:
a first bone;
a second bone; and
an apparatus of any of the preceding claims interlocked between the first and second bones.
